(19) United States
(12) Patent Application Publication
(10) Pub. No.: US 2007/0078406 A1
(43) Pub. Date: Apr. 5, 2007

(54) Safety syringe

(75) Inventor: Chien-Yuan Lee, Tamsui Town (TW)

Correspondence Address: Cooper & Dunham LLP, 1185 Avenue of the Americas, New York, NY, 10036, US

(73) Assignee: LinFair Engineering (HK) Co., Ltd.

(21) Appl. No.: 11/204,642

(22) Filed: Aug. 15, 2005

(51) Int. Cl. A61M 5/32; A61M 5/00

(52) U.S. Cl. 604/198; 604/110

(57) ABSTRACT

A safety syringe has an inner barrel, an outer barrel and a locking hub. The inner barrel is mounted removably in the outer barrel and has a front opening. The outer barrel has an inner surface and an annular detent formed in the inner surface. The locking hub is mounted on front opening and has a proximal end, a distal end, a needle mounted on the distal end and a flange formed around and protruding from the proximal end. After an injection, the inner barrel is pulled out of the outer barrel and the flange engages the annular detent. When the inner barrel separates from the outer barrel separate, the locking hub with the needle stays inside the outer barrel. The safety syringe has fewer parts so the design is simple. Less medication is wasted because the locking hub is the only component between the inner barrel and the needle.

### BACKGROUND OF THE INVENTION

1. Field of the Invention

The present invention relates to a syringe, and more particularly to a safety syringe that keeps people from being injured by the syringe after the syringe is used.

2. Description of Related Art

With reference to FIGS. 7 and 8, a conventional safety syringe comprises an inner barrel (5), a plunger (6), a retractor (7), a locking hub (9) and an outer barrel (8).

The inner barrel (5) selectively hold liquid medication and has a front opening and a rear opening.

The plunger (6) has a front and a piston (61). The piston (61) is mounted on the front of the plunger (6) and slidably in the inner barrel (5) through the rear opening. When the plunger (6) pushes into the inner barrel (5), the piston (61) pushes medication out of the inner barrel (5).

The retractor (7) is mounted removably on the front opening of the barrel (5) and has an outer edge, multiple resilience fingers (71) and a front (not numbered). The fingers (71) are formed longitudinally on the outer edge around the retractor (7) and extend away from the front of the retractor (7).

The locking hub (9) is mounted on the front of the retractor (7) and has a distal end (not numbered), a proximal end (not numbered), a fluid passage (not numbered) and a needle (91). The fluid passage is formed longitudinally through the locking hub (9). The needle (91) is mounted on and protrudes longitudinally from the distal end of the locking hub (9).

The outer barrel (8) has an inner surface (not numbered), an annular detent (81), a front opening and a rear opening. The rear opening of the outer barrel (8) allows the inner barrel (5) with needle (91) to be inserted into the outer barrel (8). The front opening of the outer barrel (8) allows the needle (91) to extend out of the outer barrel (8). The annular detent (81) is formed on the inner surface of the outer barrel (8) near the rear opening of the outer barrel (8) and selectively engages the fingers (71) on the retractor (7).

After medication is injected into a patient, the inner barrel (5) is pulled out of the outer barrel (8). The fingers (71) on the retractor (7) engage the annular detent (81) in the outer barrel (8). When the inner barrel (5) and the outer barrel (8) separate, the retractor (7) with the needle (91) is held inside the outer barrel (8) by the annular detent (81). The needle (91) cannot be used again and cannot injure anyone when the needle (91) is held inside the outer barrel (8).

However, the conventional safety syringe is complicated and wastes medication. The complexity of the conventional safety syringe causes the conventional safety syringe to be expensive to manufacture. Furthermore, the structure of the retractor (7) and the locking hub (9) hold a small but significant amount of medication even when the plunger (6) is completely depressed. The medication in the retractor (7) and the locking hub (9) is wasted and may represent a significant expense when the medication is expensive.

To overcome the shortcomings, the present invention provides a safety syringe to obviate or mitigate the aforementioned problems.

### SUMMARY OF THE INVENTION

The primary objective of the present invention is to provide a safety syringe is able to avoid a needle hurting people after the syringe is used.

The safety syringe has an inner barrel, an outer barrel and a locking hub. The inner barrel is mounted removably in the outer barrel and has a front. The outer barrel has a rear opening, an inner surface and an annular detent. The annular detent is formed on the inner surface. The locking hub is mounted on front of the inner barrel and has a proximal end, a distal end, a needle mounted on the distal end and a flange formed around and protruding from the proximal end of the locking hub. After an injection, the inner barrel is pulled out of the outer barrel, and the flange around the locking hub engages the annular detent in the outer barrel. When the inner barrel separates from the outer barrel, the locking hub with the needle stays inside the outer barrel. The safety syringe has fewer parts so its design is simple. Furthermore, less medication is wasted because the locking hub is the only component between the inner barrel and the needle.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a safety syringe in accordance with the present invention;

FIG. 2 is an exploded perspective view of the safety syringe in FIG. 1;

FIG. 3 is a side view in partial section of the safety syringe in FIG. 1;

FIG. 4 is an operational side view in partial section of the safety syringe in FIG. 1 when the inner barrel is pulled into the outer barrel;

FIG. 5 is an operational side view in partial section of the safety syringe in FIG. 1 when the flange on the locking hub engages the annular detent in the outer barrel;

FIG. 6 is an operational side view in partial section of the safety syringe in FIG. 1 when the inner barrel pulls out of the outer barrel;

FIG. 7 is an exploded perspective view of a conventional safety syringe in accordance with the prior art; and

FIG. 8 is a side view in partial section of the safety syringe in FIG. 8 when the retractor engages the outer barrel.

### DETAILED DESCRIPTION OF THE PREFERRED EMBODIMENT

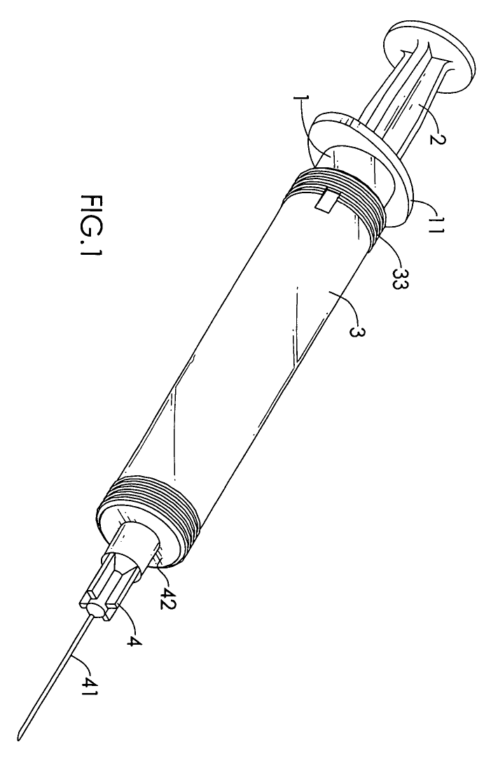

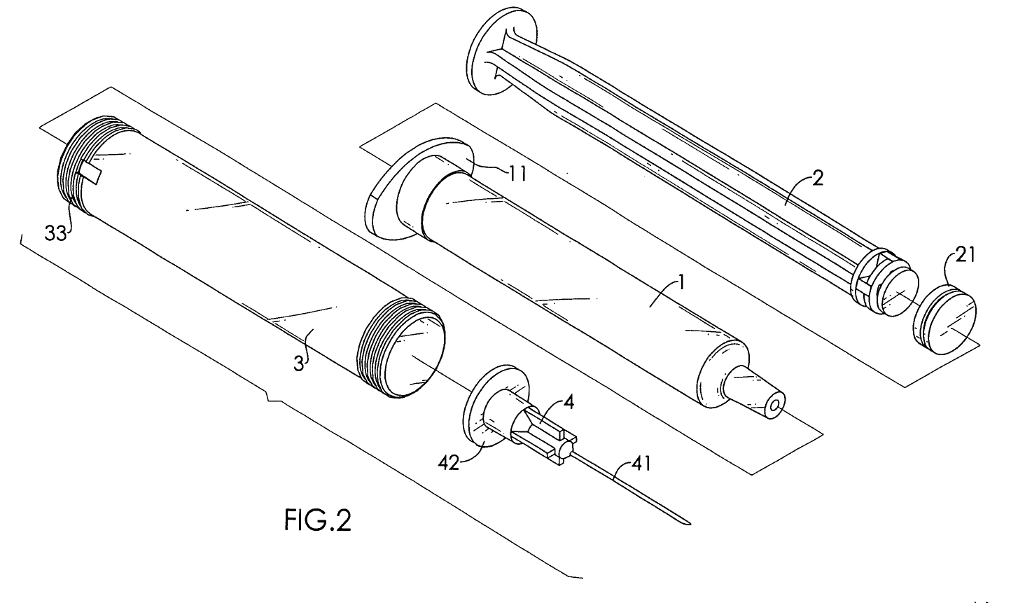

With reference to FIGS. 1, 2 and 3, a safety syringe in accordance with the present invention comprises an inner barrel (1), a plunger (2), an outer barrel (3) and a locking hub (4).

The inner barrel (1) may hold medication and has a front opening, a rear opening, an outside surface and a finger flange (11). The finger flange (11) is formed on and protrudes out from the outer surface near the rear opening and may have multiple lobes formed opposite to each other.

The plunger (2) extends slidably into the inner barrel (1) through the rear opening and has a front end and a piston (21). The piston (21) is attached to the front end of the plunger (2) and slides inside the inner barrel (1). When the plunger (2) is pushed into the inner barrel (1), medication in the inner barrel (1) is discharged.

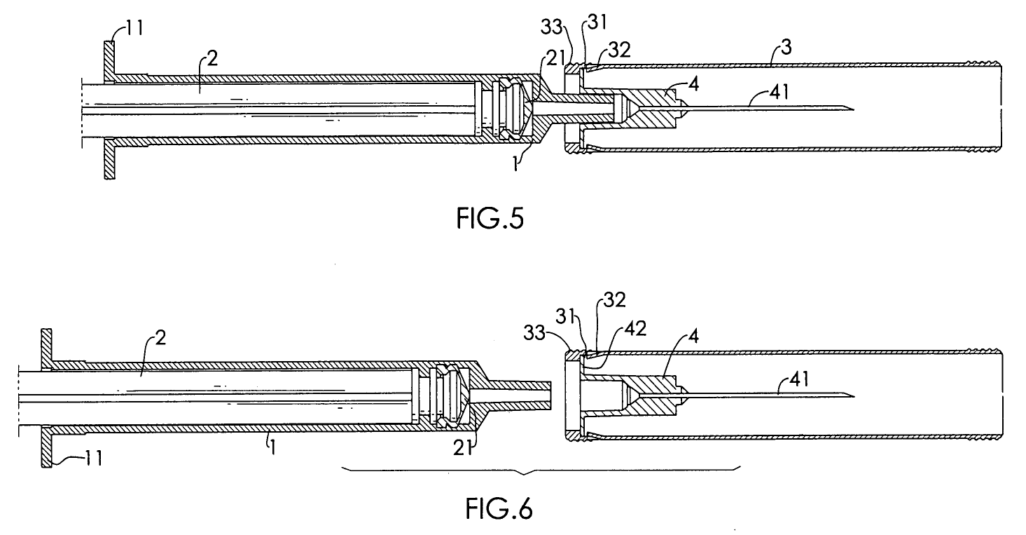

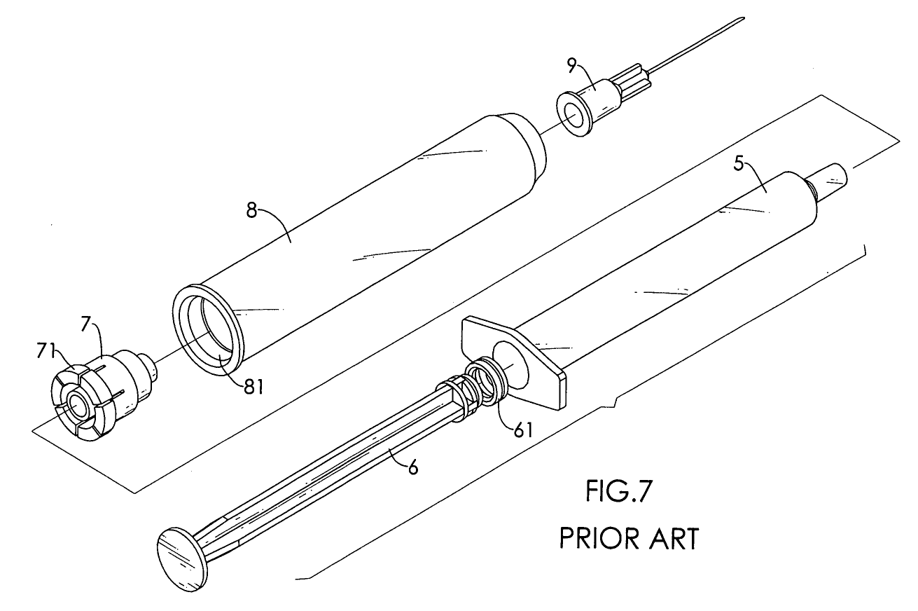

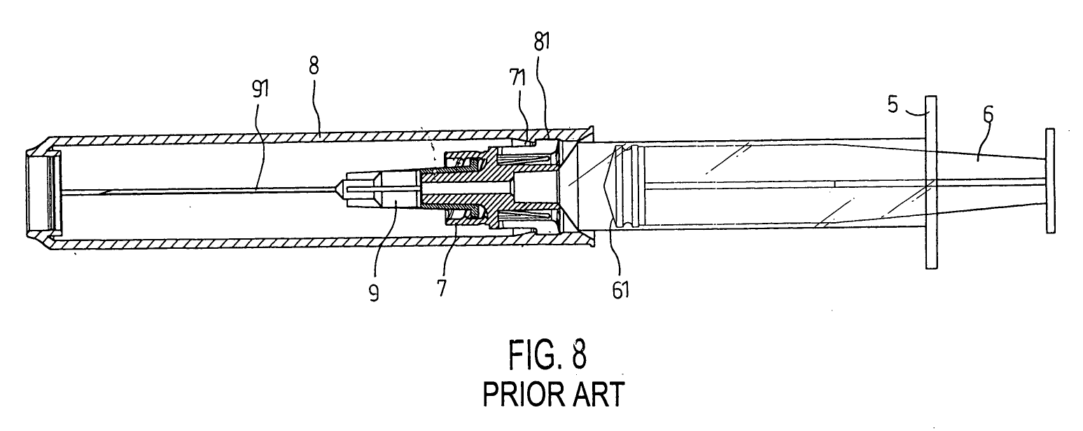

The outer barrel (3) is mounted removably on the inner barrel (1) and has a front opening, a rear opening, an outer surface, an inner surface, an annular detent (31), multiple optional resilient tabs (32) and optional grips (33). The annular detent (31) is formed in the inner surface of the outer barrel (3) near the rear opening. The resilient tabs (32) are formed on the inner surface of the outer barrel (3) adjacent to the annular detent (31) and extend into the outer barrel (2). With further reference to FIG. 6, the grips (33) are formed on the outer surface of the outer barrel (3) respectively at the front and rear openings in the outer barrel (3) so a person's hand does not slip when separating the inner barrel (1) from the outer barrel (3).

The locking hub (4) is mounted on the front opening of the inner barrel (1), selectively seats in the front opening of the outer barrel (3) and has a proximal end, a distal end, a needle (41) and a flange (42). The needle (41) is mounted on the distal end and extends out of the outer barrel (3) from the front opening in the outer barrel (3). With further reference to FIG. 5, the flange (42) is formed around and protrudes from the proximal end of the locking hub (4), selectively engages the annular detent (31) in the outer barrel (3) and is securely held in the annular detent (31) by the resilient tabs (32).

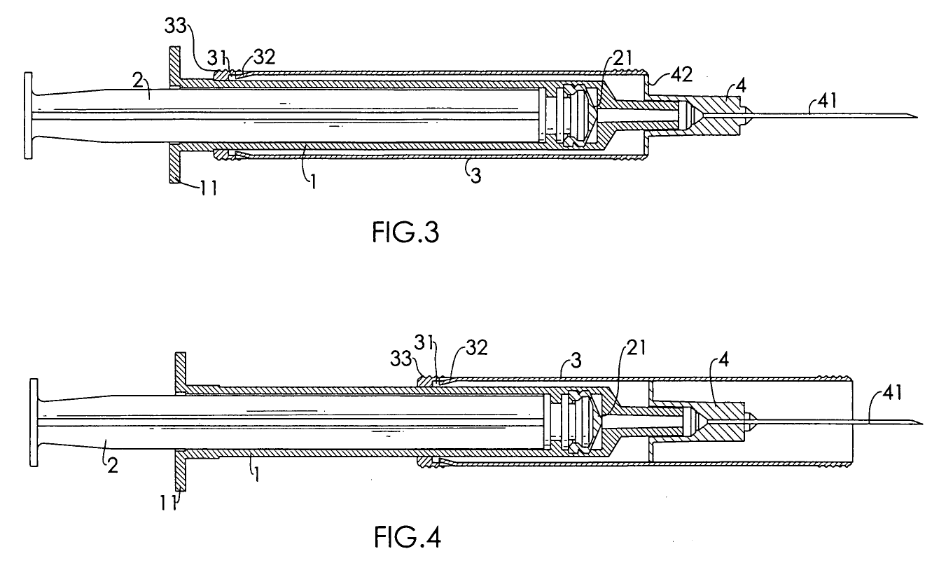

With further reference to FIG. 3, the medication only goes into the locking hub (4) so less medication is left in the syringe after the injection is done.

With further reference to FIG. 4, the inner barrel (1) is pulled out of the outer barrel (3) when the injection is done to disengage the flange (42) on the locking hub (4) from the front opening of the outer barrel (3). When the inner barrel (1) and the outer barrel (3) separate, the locking hub (4) with the needle (41) stays inside the outer barrel (3). The needle (41) cannot be used again or injure anyone.

Because the safety syringe has fewer parts, the safety syringe is easier to manufacture and assembly and costs less.

Even though numerous characteristics and advantages of the present invention have been set forth in the foregoing description together with details of the structure and function of the invention, the disclosure is illustrative only. Changes may be made in detail especially in matters of shape, size, and arrangement of parts within the principles of the invention to the full extent indicated by the broad general meaning of the terms in which the appended claims are expressed.

What is claimed is:

1. A safety syringe comprising
an inner barrel having
a front opening;
a rear opening;
an outside surface; and
a finger flange formed on and protruding out from the outer surface near the rear opening;
a plunger extending slidably into the inner barrel through the rear opening and having
a front end; and
a piston attached to the front end of the plunger and slidably mounted inside the inner barrel;
an outer barrel mounted removably on the inner barrel and having
a front opening;
a rear opening;
an outer surface;
an inner surface; and
an annular detent formed in the inner surface of the outer barrel near the rear opening;
a locking hub mounted on the front opening of the inner barrel and having
a proximal end;
a distal end;
a needle mounted on the distal end and extending out of the outer barrel from the front opening in the outer barrel; and
a flange formed around and protruding from the proximal end of the locking hub and selectively engaging the annular detent in the outer barrel.

2. The safety syringe as claimed in claim 1, wherein the outer barrel further has multiple resilient tabs formed on the inner surface of the outer barrel adjacent to the annular detent.

3. The safety syringe as claimed in claim 2, wherein the outer barrel further has grips formed on the outer surface of the outer barrel respectively at the front and the rear openings in the outer barrel.
